(19) United States
(12) Patent Application Publication
(10) Pub. No.: US 2012/0077780 A1
(43) Pub. Date: Mar. 29, 2012

(54) Fat Accumulation Inhibitor and Method of Use Thereof

(76) Inventors: Reo Tanaka, Saitama (JP); Tomoyuki Isogai, Saitama (JP); Yuko Haruta, Saitama (JP); Susumu Miura, Saitama (JP); Ken Kato, Saitama (JP); Toshimitsu Yoshioka, Saitama (JP); Hiroshi Kawakami, Saitama (JP); Satoshi Higurashi, Saitama (JP); Hiroaki Matsuyama, Saitama (JP)

(21) Appl. No.: 13/240,739

(22) Filed: Sep. 22, 2011

Related U.S. Application Data
(62) Division of application No. 12/296,222, filed on Jan. 13, 2009.

(30) Foreign Application Priority Data
Apr. 7, 2006 (JP) P2006-106164
May 31, 2006 (JP) P2006-152507

(51) Int. Cl. A61K 31/688; A61K 31/685; A61P 3/04; A61K 31/683

(52) U.S. Cl. 514/77; 514/120; 514/119

(57) ABSTRACT

The present disclosure relates to a method of inhibiting a fat accumulation for a fat cell in a subject, which includes administering a fat accumulation inhibitor for a fat cell which includes a milk-derived phospholipid as an active ingredient to the subject. The present disclosure also relates to a method of inhibiting a visceral fat accumulation in a subject, which includes administering the visceral fat accumulation inhibitor which includes a sphingosine-containing phospholipid as an active ingredient to the subject. The present disclosure also relates to a method of accelerating increase and/or inhibiting decrease of an adiponectin concentration in blood, which includes administering an agent which includes a sphingosine-containing phospholipid as an active ingredient to the blood. The present disclosure also relates to a method of inhibiting a fat accumulation for a fat cell in a subject, which includes administering a milk-derived phospholipid to a subject.

### TECHNICAL FIELD

This invention relate to a fat accumulation inhibitor for a fat cell, which comprises a milk-derived phospholipid as an active ingredient. In illustratively saying, the invention relate to a fat accumulation inhibitor for a fat cell, which comprises a milk-derived phospholipid as an active ingredient, and relates to the use of a fat accumulation inhibitor or a food or drink or the like prepared by mixing a milk-derived phospholipid, thereby providing with an action of inhibition of a fat accumulation in a fat cell, which has an action to inhibit incorporation of a lipid into a fat cell among tissue cells of mammals including human.

Also, the invention relates to a visceral fat accumulation inhibitor which comprises a sphingosine-containing phospholipid or a derivative thereof, particularly sphingomyelin, as an active ingredient, and a food or drink provided with an action of inhibition of visceral fat accumulation.

In addition, the invention relates to an agent for accelerating increase and/or inhibiting decrease of an adiponectin concentration in blood, which comprises a sphingosine-containing phospholipid or a derivative thereof, particularly sphingomyelin, as an active ingredient, and a novel food or drink provided with the action of acceleration of increase and/or inhibition of decrease of an adiponectin concentration in blood.

### BACKGROUND OF THE INVENTION

In recent years, increase of a risk of causing lifestyle-related diseases, such as obesity, hypertension, hyperlipemia, diabetes mellitus and the like due to disorder of dietary life, chronic lack of exercise, too much stress and the like, became a subject of the discussion. Advance of the symptoms of these lifestyle-related diseases results in more serious diseases such as arteriosclerosis, myocardial infarction and the like. Regarding the disorder of dietary life among the causes of lifestyle-related diseases, its large element is that dietary life of Japanese people changed to meats as the core like the case of Europeans and Americans, and therefore it became markedly high calories in comparison with conventional Japanese meal.

As a prevention of the lifestyle-related diseases caused by such a high calorie meal, roughly two methods can be considered. One is to lower concentration of a bad cholesterol and neutral fat (triglyceride) in blood, that is, to improve a lipid metabolism, and the other is to prevent obesity by inhibiting fat accumulation itself. In general, both are apt to be regarded as the same but their mechanisms are different in reality, and a medicine which improves the lipid mechanism but does not inhibit weight gain (Patent Reference 1) and a medicine which inhibits weight gain but is not concerned in the improvement of the lipid mechanism (Patent Reference 2) are respectively present.

Regarding the latter fat accumulation inhibition, the prevention and dissolution of obesity have been highlighted, not only for reasons of health but also from the cosmetic point of view. As its treatment, a drug therapy, an exercise therapy, a diet restriction and the like have been attempted. However, it is the present situation that an effect can be expected from the drug therapy, but on the contrary, it causes a necessity to take side-effects into consideration, and that the exercise therapy and diet restriction being generally carried out accompany a temporal or mental difficulty in continuously carrying out, and the success ratio is low. In addition, it cannot be ignored also that too much restriction of diet involves a risk of leading to nutritional disorders and anorexia. Under such circumstances, a fat accumulation inhibitor or a food or drink having an action of inhibition of fat accumulation, which can be ingested simply and safely in the daily dietary life, has been desired.

A phospholipid is a species of a daily ingested lipid, and it has been reported that a soybean- or egg yolk-derived phospholipid has the action of inhibition of fat accumulation (Patent Reference 3). However, a composition of the phospholipid greatly varies depending on its derivation, and regarding a milk-derived phospholipid, it has been reported only that it has an action of improvement of lipid metabolism (Patent Reference 4). That is, nothing is known about the action of inhibition of fat accumulation of the milk-derived phospholipid. In this connection, the Patent Reference 4 discloses an action of inhibition of neutral fat accumulation in a liver of the milk-derived phospholipid, but with regard to the fat accumulation in the liver, the liver cannot sufficiently treat the same because of the increase of neutral fat concentration in blood, and as a result, the surplus fat is accumulated directly. On the other hand, the one disclosed by the invention is an action of inhibition of incorporation of a lipid into a fat cell and is completely different from the above-mentioned action.

In addition, in recent years, the number of people showing morbid sates of diabetes mellitus, hypertension, hyperlipemia and arteriosclerosis as lifestyle-related diseases has been increasing accompanied by the Europeanized and Americanized life stile. Particularly, the death by cardiovascular diseases and cerebrovascular diseases occupies about one third of the cause of death, and the number thereof is increasing every year, so that a countermeasure for this is becoming a national problem. The degree of the risk of onset of these arteriosclerotic diseases is considerably increased by the accumulation of hypertension, hyperlipemia, impaired glucose tolerance and the like risk factors. This state of accumulating the risk factors is called a metabolic syndrome and has been broadly recognized.

According to an investigation on 120,000 enterprise workers in Japan, it is said that the risk of the onset of heart diseases becomes 5 times for a parson who has one risk factor among “obesity”, “hypertension”, “hyperglycemia”, “hypertriglyceride(neutral fat)emia” and “hypercholesterolemia” even when it is a mild case, and that becomes 10 times for a parson who has two of them and 31 times for a parson who jointly has 3 or 4 of them. In addition, according to an investigation by the Ministry of Health, Labor and Welfare, it is reported that the number of hypertensive patients is 39,000,000, that of hyperlipemia is 22,000,000, that of diabetes mellitus (including reserve patients) is 16,200,000 and that of obesity is 4,680,000, and these patients are increasing every year.

The metabolic syndrome is “a multiple risk factor syndrome in which accumulation of visceral fat complicates with two or more of insulin resistance, glucose metabolism disorder, dislipidemia and hypertension, based on the former, which is a morbid state of easily causing arteriosclerosis”, and the accumulation of visceral fat is certainly its basic factor. A fat tissue as a largest secretory tissue in a living body is concerned in the maintenance of homeostasis in the living body by producing various endocrine factors. However, it has been found that excess accumulation of visceral fat leads a loss of secretory balance of the endocrine factors to induce various morbid states. Particularly, the secretion quantity of a plasminogen activator inhibitor (PAI-1), a tumor necrosis factor (TNF-α), leptin and the like endocrine factors increases accompanied by the accumulation of visceral fat to induce thrombosis, insulin resistance, glucose metabolism disorder, hypertension and the like.

On the other hand, it is known that an adiponectin which is specifically secreted by the fat tissue is generally present in blood at a high concentration, but the concentration thereof decreases accompanied by the accumulation of visceral fat. Since it is known that the adiponectin has anti-diabetes, anti-arteriosclerosis, anti-inflammatory action, anti-hypertension and the like various physiological functions, acceleration of the increase of the adiponectin concentration or inhibition of the decrease of the adiponectin concentration in blood is very important for the prevention and treatment of the metabolic syndrome.

Conventionally, a drug therapy has also been carried out as a countermeasure for individual morbid state of the metabolic syndrome, but the necessity for a prescription, involvement of side effects and the like are coming into a subject of discussion. In addition, since it has been found that even when a treatment is carried out for one morbid state, it develops into a serious morbid state triggered by other morbid state, it becomes necessary to adjust secretory valance of fat cell-derived endocrine factors which are present in the upstream of these states. Because of this, it is considered that reexamination of the exercise therapy, diet restriction and the like daily life is important rather than the drug therapy for the prevention and treatment of the metabolic syndrome caused by the accumulation of the visceral fat. Accordingly, a food or drink which can be daily ingested, has high safety even when ingested over a prolonged period of time and is effective for the prevention and treatment of the metabolic syndrome caused by the accumulation of the visceral fat, has been desired.
Patent Reference 1: JP-A-2002-326946
Patent Reference 2: JP-A-2004-99539
Patent Reference 3: JP-A-10-84880
Patent Reference 4: JP-A-2001-275614

### DISCLOSURE OF THE INVENTION

### Problems that the Invention is to Solve

The objective of the invention is to provide a fat accumulation inhibitor or a food or drink for inhibition of fat accumulation, which comprises a milk-derived phospholipid as an active ingredient and can inhibit incorporation of a lipid into a fat cell by ingesting this.

In addition, the objective of the invention is to provide a visceral fat accumulation inhibitor which can be daily ingested and is effective in preventing and treating metabolic syndrome through its ingestion by inhibiting accumulation of a visceral fat or by inhibiting acceleration of increase and/or inhibition of decrease of an adiponectin concentration in blood, and an agent for accelerating increase and/or inhibiting decrease of an adiponectin concentration in blood and a food or drink to which these functions are added.

### Means for Solving the Problems

From the viewpoint of whether or not various dysfunctions in a living body can be prevented or improved by a food material which can be daily ingested, the present inventors have conducted intensive studies by taking note of a milk or milk material and found that a milk-derived phospholipid has an action to inhibit incorporation of a lipid into a fat cell, thereby resulting in the accomplishment of the invention. That is, the problems was able to be solved by providing a fat accumulation inhibitor for a fat cell, which comprises a milk-derived phospholipid as an active ingredient, or a food or drink for inhibiting a fat accumulation in the fat cell, which comprises the milk-derived phospholipid.

In addition, the inventors have conducted intensive studies on the search of a component which lowers the visceral fat considered to be a cause of the metabolic syndrome and a component which does not lower the adiponectin concentration considered to increase the risk of cardiovascular diseases when its concentration in blood is lowered, among milk components. As a result, a markedly high action of inhibition of visceral fat accumulation and action of acceleration of increase and/or inhibition of decrease of an adiponectin concentration in blood were found in a sphingosine-containing phospholipid and a derivative thereof, thereby resulting in the accomplishment of the invention.

### Advantage of the Invention

The fat accumulation inhibitor and food or drink for inhibition of fat accumulation of the invention can inhibit excess accumulation of a fat through the inhibition of incorporation of a lipid into a fat cell by ingesting them, so that they are effective for the treatment and prevention of various lifestyle-related diseases.

Also, an agent and food or drink to which an action of inhibition of visceral fat accumulation or action of acceleration of increase and/or inhibition of decrease of an adiponectin concentration in blood of the invention are added are useful for the prevention and treatment of the metabolic syndrome which is considered to be caused by accumulating the visceral fat or lowering the adiponectin in blood. In addition, as the agent and food or drink to which the action of inhibition of visceral fat accumulation and action of acceleration of increase and/or inhibition of decrease of an adiponectin concentration in blood of the invention are added, since a sphingosine-containing phospholipid and a derivative thereof, particularly sphingomyelin, are used, these are possessed of characteristics in that their supply in a large amount with relatively low cost is possible and their safety is markedly high.

### Best Mode for Carrying Out the Invention

The invention provides a fat accumulation inhibitor for a fat cell, which comprises a milk-derived phospholipid as an active ingredient, and a food or drink for inhibition of fat accumulation in a fat cell, which comprises the milk-derived phospholipid. In the fat accumulation inhibitor of the invention, a milk-derived phospholipids is used as an active ingredient. As a raw material of the milk-derived phospholipid, for example, a butter milk, a butter serum, a butter curd, a raw milk, a whey protein concentrate (WPC) and the like can be exemplified.

As a method for preparing the milk-derived phospholipid, for example, for obtaining a phospholipid fraction from a raw milk or WPC, a method for extracting with ether or acetone, a method with a water-soluble fraction containing a butter curd or butter serum, and the like conventionally known method can be exemplified. In addition, a milk derived phospholipid having an improved purity can be obtained by purifying these milk-derived phospholipid fractions through a dialysis, an ammonium sulfate fractionation, a gel filtration, an isoelectric precipitation, an ion exchange chromatography, a solvent fractionation, an ultrafiltration (UF), a microfiltration (MF) and the like techniques.

The fat accumulation inhibitor of the invention comprises a milk-derived phospholipid as an active ingredient, but the same effect can also be exerted by the use of a milk-derived phospholipid-containing composition as the milk-derived phospholipid, which is prepared from a milk material such as a butter serum, a butter milk or the like and comprises a lipid in an amount of from 20 to 90% by mass based on the total solid and a phospholipid in an amount of from 40 to 55% by mass based on the total lipid.

As a method for preparing the milk-derived phospholipid-containing composition, for example, a method in which a milk or milk material is treated with a microfiltration (MF) membrane having a pore size of from 0.1 to 2.0 μm or with an ultrafiltration (UF) membrane having a fractionating molecular weight of from 5 to 500 kDa can be exemplified.

In addition, as a method for preparing the aforementioned milk-derived phospholipid-containing composition, for example, a method in which a milk or milk material is adjusted to pH of 4.0 to 5.0 by adding an acid, thereby removing a casein protein as a precipitate, and then treating with an MF membrane having a pore size of from 0.1 to 2.0 μm or with a UF membrane having a fractionating molecular weight of from 5 to 500 kDa can be exemplified.

As the milk or milk material to be used in the preparation of the aforementioned milk-derived phospholipid-containing composition, for example, a butter milk, abutter serum and the like can be exemplified.

As a dosage form of the fat accumulation inhibitor of the invention, a tablet, a capsule, a granule, powdered materials, a powder, a syrup and the like preparations in which a milk-derived phospholipid or milk-derived phospholipid-containing composition is mixed with a stabilizer, a diluent, a binder, a disintegrant, a lubricant, a flavoring agent, a suspension agent, a coating agent and other optional drugs can be exemplified.

The food or drink for inhibition of fat accumulation of the invention can be prepared as a mixture with a food material or by blending the milk-derived phospholipid or milk-derived phospholipid-containing composition in a bread, a snack, a cake, a pudding, a drink, a fermented milk, noodles, a sausage, various types of powdered milk, a baby food and the like.

A dose of the fat accumulation inhibitor of the invention may be optionally decided by taking an object of the treatment or prevention, symptom, body weight, age, sex and the like into consideration, but it is desirable in general that approximately 1% by mass or more of the lipid to be ingested is used as the milk-derived phospholipid.

In addition, the invention provides a visceral fat accumulation inhibitor and an agent for accelerating increase and/or inhibiting decrease of an adiponectin concentration in blood, which comprises a sphingosine-containing phospholipid or a derivative thereof as an active ingredient, and a food or drink to which its functions are added. As the sphingosine-containing phospholipid or the derivative thereof, sphingomyelin is particularly desirable. This is because the sphingomyelin has a markedly high action of inhibition of the visceral fat accumulation and action of acceleration of increase and/or inhibition of decrease of an adiponectin concentration in blood.

Though the sphingomyelin is contained in a milk in a large amount of from 20 to 30% by mass in phospholipid, studies on its functions are limited to a cell level, and the finding on its physiological functions in a living body is little. Thus, its effectiveness as a component of nutrient has not so far been recognized.

Regarding an application of the sphingomyelin, an anti-inflammatory external agent, an agent for improving function of digestion and absorption of lipid, an agent for treating dysfunctional disease of intestinal movement (JP-A-5-186330, JP-A-11-269074, JP-A-2003-252765) and the like are known, but the action of inhibition of visceral fat accumulation and the action of acceleration of increase and/or inhibition of decrease of an adiponectin concentration in blood have not been revealed.

The sphingosine-containing phospholipid and a derivative thereof, particularly sphingomyelin, to be used in the invention may be purified or used as a sphingomyelin-containing phospholipid. Though the sphingomyelin is frequently contained in an animal brain and milk fat, it is desirably a milk-derived phospholipid from the viewpoint of carrying out the invention.

As a material of the milk-derived sphingomyelin, for example, a raw milk, a whey protein concentrate (WPC), a butter curd, a butter serum and the like can be exemplified. As a method for preparing the milk-derived sphingomyelin, for example, a method for extracting with ether or acetone for obtaining a sphingomyelin-containing phospholipid fraction from a raw milk, WPC or the like (JP-A-3-47192), a method which uses a water-soluble fraction containing a butter curd or a butter serum, and the like conventionally known method can be exemplified. The sphingomyelin content of the fraction obtained by employing these materials and methods is about 28% by mass and about 9% by mass, respectively.

In addition, sphingomyelin having improved purity can be obtained by purifying the aforementioned sphingomyelin-containing phospholipid fraction through a dialysis, an ammonium sulfate fractionation, a gel filtration, an isoelectric precipitation, an ion exchange chromatography, a solvent fractionation, an ultrafiltration (UF), a microfiltration (MF) and the like techniques.

The sphingomyelin or sphingomyelin-containing phospholipid obtained by the above-mentioned method can be made into liquid, powder, tablets and the like optional forms and can be directly administered orally. In addition, a phospholipid composition containing not only the sphingomyelin but also an effective amount of phosphatidylcholine defined as a necessary amount of human nutrition may be used.

As the dosage forms of the visceral fat accumulation inhibitor and agent for accelerating increase and/or inhibiting decrease of an adiponectin concentration in blood of the invention, a tablet, a capsule, a granule, powdered materials, a powder and the like can be exemplified.

In addition, as a food or drink to which the action of inhibition of visceral fat accumulation and action of acceleration of increase and/or inhibition of decrease of an adiponectin concentration in blood were added, those in which a sphingosine-containing phospholipid and a derivative thereof, particularly sphingomyelin, is contained in a milk, a milk drink, a coffee drink, a juice, a jelly, a biscuit, a bread, a noodle, a sausage and the like food or drink, and various types of powdered milk, as well as a nutritious composition aimed at a suckling, a baby, a low birth weight infant and the like can be exemplified. Since these can be daily ingested and have the action of inhibition of visceral fat accumulation and action of acceleration of increase and/or inhibition of decrease of an adiponectin concentration in blood, they are useful in preventing and treating the metabolic syndrome caused by the increase of visceral fat and decrease of the adiponectin in blood.

In order to exert the action of inhibition of visceral fat accumulation and action of acceleration of increase and/or inhibition of decrease of an adiponectin concentration in blood by the agent and food or drink to which the action of inhibition of visceral fat accumulation and action of acceleration of increase and/or inhibition of decrease of an adiponectin concentration in blood were added, the blending amounts and the like may be adjusted such a manner that from 0.1 to 5,000 mg per day of a sphingosine-containing phospholipid and a derivative thereof, particularly sphingomyelin, can be ingested in the case of adult.

The following describes the invention further in detail with reference to examples and test examples, but these are simple exemplifications so that the invention is not restricted thereby.

### EXAMPLES

### Example 1

20% by mass solution of a butter serum powder (SM 2, manufactured by Corman) was prepared, and the pH was adjusted to 4.5 by adding 5 M hydrochloric acid. This solution was allowed to stand at 50° C. for 1 hour to effect precipitation of a casein protein. This precipitate was removed by using a filter press, and the thus obtained aqueous solution was treated with an MF membrane (manufactured by SCT) having a pore size of 1.4 μm to obtain a concentrated liquid fraction. This concentrated liquid fraction was subjected to a freeze-drying treatment to obtain a milk-derived phospholipid-containing composition. This milk-derived phospholipid-containing composition contained, per the total solid, 56% by mass of a lipid, 25% by mass of a protein, 13% by mass of a carbohydrate and 6% by mass of an ash, and 48% by mass of the total lipid was a phospholipid.

### Example 2

15% by mass solution of a butter milk powder (manufactured by Snow Brand Milk Products Co., Ltd.) was prepared, and the pH was adjusted to 4.5 by adding 1 M hydrochloric acid. This solution was allowed to stand at 40° C. for 30 minutes to effect precipitation of a casein protein. After removing this precipitate by using a clarifier, the thus obtained supernatant was treated with an MF membrane (manufactured by SCT) having a pore size of 0.1 μm to obtain a concentrated liquid fraction. This concentrated liquid fraction was subjected to a freeze-drying treatment to obtain a milk-derived phospholipid-containing composition. This milk-derived phospholipid-containing composition contained, per the total solid, 50% by mass of a lipid, 27% by mass of a protein, 16% by mass of a carbohydrate and 7% by mass of an ash, and 40% by mass of the total lipid was a phospholipid.

### Test Example 1

### Verification of Action of Inhibition of Fat Accumulation in Fat Cell

Using the milk-derived phospholipid-containing compositions obtained in Examples 1 and 2, an action of inhibition of fat accumulation in a fat cell was verified. The test was carried out by allowing a mouse-derived precursor fat cell 3T3-L1 to differentiate into a fat cell, and comparing the amount of neutral fat in the fat cell. The 3T3-L1 cells suspended in Dulbecco's modified Eagle's medium (DMEM) comprising 10% by mass of fetal bovine serum were inoculated into a 6 well plate and cultured at 37° C. for 10 days. Thereafter, the 3T3-L1 cells were differentiated into a fat cell by culturing for 2 days using a medium containing 0.25 μM dexamethasone, 0.5 mM isobutylmethylxanthine and 10 nM insulin. The thus obtained fat cell was cultured for about 1 week using the medium in which the dexamethasone and isobutylmethylxanthine were removed and the 10 nM insulin alone and a phospholipid-containing composition (final phospholipid concentration, from 5 to 25 μg/ml) were added therein, and an amount of the neutral fat was measured. In addition, the medium containing the 10 nM insulin alone which was cultured for about 1 week was used as a control.

The results are shown in Table 1. In comparison with the control, the significant action of inhibition of fat accumulation was found in Examples 1 and 2 as the invention. Also, the action of inhibition of fat accumulation was found also in α-Lipid and β-Lipid which were commercially available milk-derived phospholipids, but their effects were lower than those of Examples 1 and 2. In addition, it was revealed that this action of inhibition of fat accumulation depends on the concentration of the milk-derived phospholipid. On the other hand, the action of inhibition of fat accumulation in the fat cell was found also in the case that the soybean lecithin or egg yolk lecithin, as the soybean- or egg yolk-derived phospholipids, was added, but the effect was lower than the case that the milk-derived phospholipid of Examples 1 and 2 was added. This shows that the action of inhibition of fat accumulation in a fat cell of milk-derived phospholipid is superior to the action of inhibition of fat accumulation in a fat cell of other phospholipids.

TABLE 1

| | | Phospholipid | Fat accumulation |
|---|---|---|---|
| Substances | | concentration | inhibition ratio |
| administered | | [μg/ml] | [%] |
| 1. | Control | 0.0 | — |
| 2. | Commercially | 10.0 | 9.2 |
| | available product | | |
| | (α-Lipid) | | |
| 3. | Commercially | 10.0 | 9.8 |
| | available product | | |
| | (β-Lipid) | | |
| 4. | Soybean lectin | 10.0 | 7.1 |
| 5. | Egg yolk lectin | 10.0 | 6.7 |
| 6. | Example 1 | 5.0 | 9.0 |
| 7. | Example 1 | 10.0 | 12.7 |
| 8. | Example 1 | 20.0 | 16.2 |
| 9. | Example 2 | 10.0 | 14.8 |

### Test Example 2

### Verification of Action of Inhibition of Fat Accumulation in Fat Cell in Living Body

An animal test was carried out using 5 animals per group of Wistar-type rat of 6 weeks of age by a group in which a fat calorie ratio was set to about 10% by mass with a lard and a corn oil (ordinary diet group), a group in which a high-fat fat was fed by setting the fat calorie ratio to 50% by mass (control group), and groups in which a part of the high-fat fat was replaced by each phospholipids (invention group, α-Lipid group, soybean lecithin group, egg yolk lecithin group). After 1 week of preliminary rearing, rearing was carried out for 4 weeks by freely providing the diet based on each condition and freely providing water every day. Thereafter, a body weight gain, a feed efficiency, a fat tissue mass in epididymis fat and a fat tissue mass in perirenal fat were measured.

The results are shown in Table 2. Predominant difference in feed ingestion amount was not found among groups. In comparison with the control group, the invention group showed significantly low values in terms of the body weight gain and fat tissue masses in perirenal and peri-testicular. In addition, the inventive group also showed low values in terms of the respective indexes in comparison with other phospholipid groups. This shows that the milk-derived phospholipid-containing composition obtained in Example 1 has excellent action of inhibition of accumulation in a fat cell.

TABLE 2

| | Body | Feed | Perirenal | Peri-testicular |
|---|---|---|---|---|
| | weight | efficiency | fat tissue | fat tissue |
| | gain [g] | [%] | [g] | [g] |
| Ordinary diet group | 116.0 | 0.16 | 6.2 | 5.7 |
| Control group (high | 132.5 | 0.26 | 10.5 | 9.8 |
| fat diet) | | | | |
| α-Lipid group | 126.2 | 0.25 | 9.5 | 8.9 |
| Soybean lectin group | 125.4 | 0.25 | 9.4 | 9.1 |
| Egg yolk lectin group | 127.1 | 0.25 | 9.7 | 9.2 |
| Invention group | 120.7 | 0.23 | 7.8 | 7.4 |

### Example 3

50 g of the milk-derived phospholipid-containing composition of Example 1 was dissolved in 4950 g of deionized water, heated to 50° C. and then mixed under stirring at 6000 rpm for 30 minutes using a TK homo-mixer (TK ROBO MICS; manufactured by Tokushu Kika Kogyo) to obtain a milk-derived phospholipid-containing composition solution comprising 250 mg/100 g of the milk-derived phospholipid-containing composition. 4.0 kg of this milk-derived phospholipid-containing composition solution was blended with 5.0 kg of a casein, 5.0 kg of a soybean protein, 1.0 kg of a fish oil, 3.0 kg of a perilla oil, 18.0 kg of a dextrin, 6.0 kg of a mineral mixture, 1.95 kg of a vitamin mixture, 2.0 kg of an emulsifier, 4.0 kg of a stabilizer and 0.05 kg of a fragrance, packed in a 200 ml capacity retort pouch and sterilized at 121° C. for 20 minutes using a retort sterilizer (a first order pressure container, TYPE: RCS-4CRTGN, manufactured by Hisaka Works) to obtain 50 kg of the liquid nutriment composition for inhibition of fat accumulation of the invention.

### Example 4

10 g of the milk-derived phospholipid-containing composition of Example 1 was dissolved in 700 g of deionized water, heated to 50° C. and then mixed under stirring at 9500 rpm for 30 minutes using an ultra-disperser (ULTRA-TURRAX T-25; manufactured by IKA Japan). 40 g of a sorbitol, 2 g of an acidulent, 2 g of a fragrance, 5 g of a pectin, 5 g of a milk serum protein concentrate, 1 g of calcium lactate and 235 g of deionized water were added to this solution, and this was mixed under stirring, and then packed in a 200 ml capacity drink pouch and sterilized at 85° C. for 20 minutes, and then sealed to prepare 5 bags (200 g) of the gelatinous food for inhibition of fat accumulation of the invention. In all cases of the gelatinous food for inhibition of fat accumulation obtained in this manner, precipitate and the like were not found and storange flavor was not felt. In this connection, a milk-derived phospholipid-containing composition was contained in this gelatinous food for inhibition of fat accumulation in an amount of 100 mg per 100 g.

### Example 5

After dissolving 2 g of an acidulant in 700 g of deionized water, 10 g of a milk-derived phospholipid-containing composition was dissolved therein and this was heated to 50° C. and then mixed under stirring at 9500 rpm for 30 minutes using an ultra-disperser (ULTRA-TURRAX T-25; manufactured by IKA Japan). 100 g of a maltitol, 20 g of a reduced starch syrup, 2 g of a fragrance and 166 g of deionized water were added thereto, and then that was packed in a 100 ml capacity glass bottle and sterilized at 90° C. for 15 minutes, and then sealed to prepare 10 bottles (100 ml) of a drink for fat accumulation inhibition. In all cases of the drink for fat accumulation inhibition obtained in this manner, precipitate was not found and strange taste was not felt. In this connection, a milk-derived phospholipid-containing composition was contained in this drink for fat accumulation inhibition in an amount of 250 mg per 100 g.

### Example 6

The milk-derived phospholipid-containing composition of Example 1 was dissolved in 98 kg of deionized water, heated to 50° C. and then mixed under stirring at 3600 rpm for 40 minutes using a TK homo-mixer (MARK II model 160; manufactured by Tokushu Kika Kogyo) to obtain a milk-derived phospholipid-containing composition solution comprising 80 mg/100 g of the milk-derived phospholipid-containing composition. 10 kg of this milk-derived phospholipid-containing composition solution was blended with 12 kg of a soybean cake, 14 kg of a fat-free milk, 4 kg of a soybean oil, 2 kg of a corn oil, 23.2 kg of a palm oil, 14 kg of a cornstarch, 9 kg of a wheat flour, 2 kg of a wheat bran, 5 kg of a vitamin mixture, 2.8 kg of a cellulose and 2 kg of a mineral mixture, and sterilized at 120° C. for 4 minutes to produce 100 kg of the dog breeding feed for inhibition of fat accumulation of the invention. In this connection, a milk-derived phospholipid-containing composition was contained in this dog breeding feed for inhibition of fat accumulation in an amount of 8 mg per 100 g.

### Example 7

A protease was allowed to react with 10% by mass aqueous solution of a whey protein concentrate (WPC), and the thus obtained reaction liquid was extracted with chloroform-methanol (2:1) and then concentrated, and further extracted with acetone to obtain a complex lipid fraction. Next, this complex lipid fraction was treated with a fluorosilyl column chromatography and subjected to a stepwise extraction with chloroform-methanol solution to obtain a phospholipid fraction. This phospholipid fraction was treated with a silica-gel chromatography and subjected to a stepwise extraction with chloroform-methanol solution, and the product was freeze-dried to obtain sphingomyelin. When this sphingomyelin was treated with a thin-layer chromatography and then subjected to a development of color with Dittmer's reagent and measured by a densitometry method, the sphingomyelin content was 95.2% by mass. The sphingomyelin obtained in this manner can be used directly as a visceral fat accumulation inhibitor and an agent for accelerating increase and/or inhibiting decrease of an adiponectin concentration in blood.

### Test Example 3

### Verification of Action of Acceleration of Increase and/or Inhibition of Decrease of Adiponectin Concentration in Blood

Using the sphingomyelin obtained in Example 7, an action of acceleration of increase and/or inhibition of decrease of an adiponectin concentration in blood was verified. An animal test was carried out using 8 animals per group by a group in which a high fat feed blended with sphingomyelin was fed (sphingomyelin diet group) and a group in which a high fat feed not blended with sphingomyelin was fed (high fat diet group). The high fat diet was prepared using a milk casein as a protein source and a butter oil as a lipid source. In the animal test, a feed was provided until the 4^{th} week after rearing, and thereafter the group was divided into 5 groups, and the high fat feed or sphingomyelin-blended high fat feed was provided until the 8^{th} week. Blood collection was carried out on the 4^{th} week and on the 8^{th} week, and an adiponectin concentration in blood was measured using a Mouse/Rat Adiponectin ELISA Kit (manufactured by Otsuka Pharmaceutical Co., Ltd.).

The results are shown in Table 3. According to this, it was revealed that the adiponectin concentration in blood in the high fat diet group decreases with the lapse of time, while it increases in the sphingomyelin diet group. That is, it was found that increase of the adiponectin concentration in blood is accelerated or its decrease is inhibited by the ingestion of sphingomyelin.

TABLE 3

Adiponectin
concentration in
blood (μg/ml)

| Feed group | 4 weeks | 8 weeks |
|---|---|---|
| High fat diet | 11.54 | 11.07 |
| Sphingomyelin (Example | 10.88 | 15.65 |
| 7) diet | | |

### Test Example 4

### Verification of Action of Visceral Fat Accumulation Inhibition

Using the sphingomyelin obtained in Example 7, an action of inhibition of visceral fat accumulation was verified. An animal test was carried out using 8 animals per group by a group in which a high fat feed blended with sphingomyelin was fed (sphingomyelin diet group) and a group in which a high fat feed not blended with sphingomyelin was fed (high fat diet group). In the animal test, the high fat feed was provided until the 4^{th} week after rearing, and thereafter the group was divided into 5 groups, and the high fat feed or sphingomyelin-blended high fat feed was provided until the 8^{th} week. By carrying out dissection on the 8^{th} week, the amount of visceral fats (mesenteric, peri-testicular, perirenal, posterior abdominal) was measured.

The results are shown in Table 4. According to this, it was revealed that, in comparison with the amount of the visceral fat of the high fat diet group, the sphingomyelin diet group generally shows low values. That is, it was found that the accumulation of the visceral fat is inhibited by ingesting sphingomyelin.

TABLE 4

| | Amount of visceral fat (g) | | | |
|---|---|---|---|---|
| | | | | posterior |
| Feed group | Mesenteric | peri-testicular | Perirenal | abdominal |
| High fat diet | 2.96 | 3.68 | 0.98 | 2.56 |
| Sphingomyelin | 2.19 | 3.22 | 0.74 | 2.11 |
| (Example 7) diet | | | | |

### Example 8

Materials were mixed based on the mixture shown in Table 5, and that was granulated and then packed in a capsule to produce capsules to which an action of inhibition of visceral fat accumulation or an action of acceleration of increase and/or inhibition of decrease of an adiponectin concentration in blood was added.

TABLE 5

| | (% by mass) |
|---|---|
| Sphingomyelin (Example 7) | 20 |
| Lactose | 24.5 |
| Soluble starch | 55 |
| Magnesium stearate | 0.5 |

### Example 9

Materials were mixed based on the mixture shown in Table 6 and that was packed in a container and then heat-sterilized to produce a drink for inhibition of visceral fat accumulation or a drink for acceleration of increase and/or inhibition of decrease of an adiponectin concentration in blood.

TABLE 6

| | (% by mass) |
|---|---|
| Sphingomyelin (Example 7) | 2.5 |
| Sugar | 7.5 |
| Citric acid | 0.6 |
| Apple juice | 10 |
| Water | 79.4 |

1-14. (canceled)

15. A method of inhibiting a fat accumulation for a fat cell in a subject, which comprises administering a fat accumulation inhibitor for a fat cell which comprises a milk-derived phospholipid as an active ingredient to the subject.

16. The method according to claim 15, wherein the fat accumulation inhibitor comprises an effective amount of a milk-derived phospholipid composition for inhibiting fat accumulation,
wherein the milk-derived phospholipid composition comprises a lipid in an amount of from 20 to 90% by mass based on the total composition, and a phospholipid in an amount of from 40 to 55% by mass based on the total lipid.

17. The method according to claim 15, wherein the milk-derived phospholipid composition comprises an ash in an amount of at least 7% by mass based on the total composition.

18. The method according to claim 15, wherein the milk-derived phospholipid-containing composition is derived from a milk material.

19. The method according to claim 18, wherein the milk-derived phospholipid-containing composition is obtained by treating a milk or milk material with a microfiltration (MF) membrane having a pore size of from 0.1 to 2.0 μm or with an ultrafiltration (UF) membrane having a fractionating molecular weight of from 5 to 500 kDa.

20. The method according to claim 18, wherein the milk-derived phospholipid-containing composition is obtained by adjusting a milk or milk material to pH of 4.0 to 5.0 by adding an acid, thereby removing a casein protein as a precipitate, and then treating with an MF membrane having a pore size of from 0.1 to 2.0 μm or with a UF membrane having a fractionating molecular weight of from 5 to 500 kDa.

21. The method according to claim 18, wherein the milk or milk material is a butter serum or a butter milk.

22. A method of inhibiting a visceral fat accumulation in a subject, which comprises administering the visceral fat accumulation inhibitor which comprises a sphingosine-containing phospholipid as an active ingredient to the subject.

23. The method according to claim 22, wherein the visceral fat accumulation inhibitor comprises an effective amount of a sphingosine-containing phospholipid composition comprising sphingomyelin in an amount of at least 95% by mass based on the total composition.

24. The method according to claim 23, wherein the sphingosine-containing phospholipid composition is derived from a milk material.

25. The method according to claim 22, wherein the sphingosine-containing phospholipid is sphingomyelin.

26. A method of accelerating increase and/or inhibiting decrease of an adiponectin concentration in blood, which comprises administering an agent which comprises a sphingosine-containing phospholipid as an active ingredient to the blood.

27. The method according to claim 26, wherein the agent comprises an effective amount of a sphingosine-containing phospholipid composition comprising a sphingomyeline in an amount of at least 95% by mass based on the total agent.

28. The method according to claim 27, wherein the sphingosine-containing phospholipid composition is derived from a milk material.

29. The method according to claim 26, wherein the sphingosine-containing phospholipid is sphingomyelin.

30. A method of inhibiting a fat accumulation for a fat cell in a subject, which comprises administering a milk-derived phospholipid to a subject.

31. The method of claim 30, wherein the phospholipid is obtained by
treating a milk or milk material with a microfiltration (MF) membrane having a pore size of from 0.1 to 2.0 μm or with an ultrafiltration (UF) membrane having a fractionating molecular weight of from 5 to 500 kDa to obtain a milk-derived phospholipid.

32. The method of claim 31, wherein the phospholipid is obtained by
removing a casein protein as a precipitate by adjusting a milk or milk material to pH of 4.0 to 5.0 by adding an acid prior to the treatment with the MF membrane or UF membrane.

33. The method of claim 30, wherein the phospholipid is derived from butter serum or a butter milk.
